(19) United States
(12) Patent Application Publication
(10) Pub. No.: US 2014/0356288 A1
(43) Pub. Date: Dec. 4, 2014

(54) Topical Examination Compositions and Methods

(71) Applicants: Melissa Kirkpatrick, Sarasota, FL (US); Regina Lim, Holly Hill, FL (US)

(72) Inventors: Melissa Kirkpatrick, Sarasota, FL (US); Regina Lim, Holly Hill, FL (US)

(73) Assignee: MELISSA KIRKPATRICK, Sarasota, FL (US)

(21) Appl. No.: 14/289,390

(22) Filed: May 28, 2014

Related U.S. Application Data
(60) Provisional application No. 61/828,377, filed on May 29, 2013.

(51) Int. Cl. A61K 9/00; A61K 47/10; A61K 47/22; A61K 47/46

(52) U.S. Cl. CPC A61K 9/0041; A61K 47/46; A61K 47/10; A61K 47/22
USPC 424/9.1

(57) ABSTRACT

Topical compositions for examining the tissue under human breasts for lumps, nodules, knots or thickening comprising: (a) a “Pro Breast Health Complex” consisting essentially of (i) Green Tea Extract, (ii) Vitamin D, or a derivative thereof, (iii) Vitamin E, or a derivative thereof, and (iv) Oenothera Biennis Oil; and (b) an Adjuvant Aromatherapy Blend that is complementary to the PBH Complex that comprises (i) Rosa Damascena Seed Oil, (ii) Lavandula Angustifolia Oil, and (iii) Cymbopogon Schoenanthus Oil. The composition does not contain parabens and does not contain petroleum-based surfactants.

### BACKGROUND OF THE INVENTION

The importance of breast self-examination in the early diagnosis and treatment of breast cancer is widely recognized in the medical community. Breast self-examination helps a woman (or man) to become familiar with how her (or his) breasts look and feel, so she/he can alert her/his healthcare professional if there are any changes. In many instances, breast cancer is detected after a woman (or man) first feels a lump during breast self-exam.

U.S. Pat. No. 5,873,367, now expired, is directed to a method of breast examination. According to its Abstract, this patent discloses a method for examining the breast tissue of an individual for the presence of lumps or a change in texture, which method includes the steps of: applying to the skin of the individual in the area of the breast tissue while the skin is substantially dry an amount of an aqueous emulsion or gel composition effective to lubricate the skin; and feeling the skin of the individual by hand for a lump or thickening in the underlying tissue; whereby the lubricating effect of the emulsion or gel composition increases the tactile sensation of the texture of the breast tissue.

Despite the recognized clinical importance of breast self-examination, there exists only a few creams or lotions that are marketed for this specific purpose. The formulation of a topical product that facilitates palpation is complex, requiring a balance between sensorial and aesthetic properties with physical-chemical parameters, including viscosity. Upon application, a product must deposit a thin, comfortable film that allows for easy spreading and sufficient playtime to perform palpation without runoff. The latter presents a significant obstacle in the design of an efficacious breast self-examination product intended for use in a wet environment (i.e., a shower or bath).

Another challenge in formulating a topically-applied breast self-examination product, particularly one designed for use in a wet environment, is to exclude petroleum derived ingredients, in particular ethoxylates, and certain preservatives (i.e., parabens) that have, under certain conditions, been reported to be associated with increased risk of developing cancer and other health-related complications.

The topically-applied breast self-examination product of the present invention meets these formulating challenges. In a formulation that is free of petroleum-derived surfactants and parabens, the present invention provides a topical product specifically designed for use in a shower or bath that deposits a thin, comfortable film, allowing for easy spreading and sufficient playtime to perform palpation without runoff.

The scientific community recognizes the potential breast health benefits, including in terms of reduced risk of developing breast cancer, of maintaining and receiving on a regular basis certain doses of vitamins and antioxidants. For example, research suggests that women with low levels of vitamin D have a higher risk of breast cancer and that Vitamin D may play a role in regulating normal breast cell growth, including by stopping the uncontrolled division of breast cells that can manifest as carcinoma. The potential role of vitamin E in protecting women from heart attack, stroke, or cancer continues to be investigated. In addition, polyphenols, including in particular epigallocatechin-3-gallate (EGCG) found in green tea extract, have been reported to play a role in preventing cell damage that can lead to breast cancer. Recent research, including from the U.S. National Institutes of Health National Cancer Institutes, reports that the biochemicals in green tea exert a positive role in regulating estrogen metabolism, and thereby decreasing the risk of breast cancer. See, e.g., L. Moreira et al, “Quercetin and epigallocatechin gallate inhibit glucose uptake and metabolism by breast cancer cells by an estrogen receptor-independent mechanism”, Exp. Cell Res: 2013 (May 9), S0014-4827.

Accordingly, one aspect of the present invention is to provide polyphenols, Vitamin D, and Vitamin E as part of a regular regimen of breast self-examination.

Additionally, the clinical literature recognizes the therapeutic value of aromatherapy ingredients. Accordingly, a second aspect of the present invention is directed to providing aromatherapy during breast self-examination.

Testicular cancer, the most common cancer in males between the ages of 15 and 34, is treatable and curable, especially if detected early. Another aspect of the present invention is providing a topically applied creme that can be used in examination for signs of testicular cancer.

A further aspect of the present invention is directed to a topically applied creme that can be used in physical examination for signs of muscle strain or tear.

### SUMMARY OF THE INVENTION

The present invention relates to a moisturizing aromatherapy creme useful in breast self examination containing a Pro Breast Health Complex consisting essentially of, and in a particularly preferred embodiment, consisting of, each of green tea extract, Vitamin D (or derivatives thereof), Vitamin E (or derivatives thereof), and Oenothera Biennis (Evening Primrose) Oil. In preferred embodiments, in addition to the Pro Breast Health Protective Complex, the moisturizing aromatherapy creme of the present invention also contains an Adjuvant Aromatherapy Complex containing at least essential oils of rose, lavender and lemongrass.

While a preferred aspect of the present invention is directed to a natural, moisturizing aromatherapy creme useful in breast self-examination (BSE), the moisturizing aromatherapy creme can also be used in clinical breast examination (i.e., by a licensed medical professional), and also examination for signs of testicular cancer in men. The moisturizing aromatherapy creme useful in breast self examination of the present invention can be used both by women and men. Aspects of the invention directed to use of the moisturizing aromatherapy creme for breast self-examination are referred to herein below as the natural, BSE moisturizing aromatherapy creme.

The natural, BSE moisturizing aromatherapy creme is “natural” by which is meant it forms an emulsion without the use of petroleum-based surfactants, namely ethoxylates, and is made with organic ingredients in compliance with the National Organic Program, 7 C.F.R. §205.605.

In a preferred embodiment of the present invention, the natural, BSE moisturizing aromatherapy creme is applied during or immediately after bathing or showering.

### DETAILED DESCRIPTION OF THE INVENTION

As used herein and unless otherwise expressly noted or required by the context, all percentages refer to percentages by weight (wt-%).

As used herein in connection with a measured quantity, for example weight, “about” refers to that variation in the measured quantity as would be expected by one skilled in the art exercising a level of care commensurate with the objective of the measurement and the equipment used, and includes uncertainties that may be introduced by mathematical rounding errors.

As used in the present application, by the term “breast examination” is meant a method whereby through palpation a lump, nodule, knot or thickening inside the breast or underarm area is detected. By the term “breast self-examination” is meant breast examination performed by a woman herself.

“Breast examination”, whether by a trained medical professional, or a woman herself, may be conducted either standing or lying, and carefully feeling the entire breast area from the collarbone to the bottom of the ribcage, and from the armpit to the breastbone.

Breast self-examination conducted by palpation within the scope of the present invention can be performed in or after a shower or bath on wet or damp skin, or on a “dry” breast in a standing position, preferably in front of a mirror in combination with visual self-examination or in a recumbent position (lying down).

Whether standing or lying down, a woman performs breast self-examination by moving the fleshy pads on the volar side of her three middle fingertips (same side as the palms) across the entirety of each breast, in a circular pattern, beginning from the outside and moving towards the center, checking the entire breast and armpit area to the bottom of the ribcage. Preferably, the woman presses the pads of the fingertips of her three middle right fingers around her left breast, and repeats this procedure pressing the pads of the fingertips of her three middle left fingers around her right breast.

In each successive circular motion, light pressure is applied to feel the tissue close to the skin surface. Medium pressure is applied to feel deeper, and firm pressure is used to feel the tissue close to the breastbone and ribs. Each pressure level should be used to feel each section of breast tissue before moving on to the next section.

Because breast tissue spreads out along the chest wall when lying down, some women may prefer, and some medical professionals may recommend, conducting breast self-examination while lying down. When conducting breast self-examination in a recumbent position, a woman may prefer to place a pillow under her right shoulder and right arm behind her head. Using her left hand, the woman moves the pads of her fingertips around her right breast gently in small circular motions covering the entire breast area and armpit. A similar procedure is performed on the left breast. A woman places a pillow under her left shoulder and left arm behind her head. Using her right hand, the woman moves the pads of her fingertips around her left breast gently in small circular motions covering the entire breast area and armpit.

As will be appreciated by the skilled artisan, other methods may be used to conduct breast self-examination and are within the scope of the present invention. For example, breast self-examination may be conducted by a pattern of up and down lines or wedges, starting at the outer edge, moving the fingers in towards the nipple and then back out to the edge.

In a preferred embodiment, in pre-menopausal women, who are not pregnant, breast self-examination is performed one week after the menstrual period begins. It is during this time that breast tissue is least likely to be swollen or tender.

In other embodiments, including for women who experience irregular menstrual cycle, or in women who have stopped menstruating (i.e., due to menopause or hysterectomy), breast self-examination is done on or about the same of each day month (e.g., the first day of each month).

In another embodiment, directed to women who are pregnant or breast-feeding, breast self-examination can also be done regularly, once per month. Mothers who are lactating can examine their breasts after a feeding or after using a breast pump.

An essential component of the natural, BSE moisturizing aromatherapy creme of the present invention is a four-part Pro Breast Health (PBH) Complex consisting of four ingredients, each as described below: green tea extract, Vitamin D (or derivatives thereof); Vitamin E (or derivatives thereof); and Oenothera Biennis (Evening Primrose) Oil.

One embodiment of the natural, moisturizing aromatherapy creme of the present invention that is useful in breast examination (both by a woman or a physician or other healthcare provider) can also be used in physical examination (both in a clinical setting and by a man himself) for signs of testicular cancer, namely lumps or bumps detected by tactile examination. This embodiment can also be used in physical examination for signs of muscle strain or tear. Physical examination in accordance with these embodiments of the present invention may be conducted by a trained allied health professional, including a physician, a nurse, a nurse practitioner, or a therapist.

As used in the present application, examination for signs of testicular cancer involves feeling the underneath the skin of the scrotal sac for hard lumps or smooth rounded bumps on the testes. In one embodiment of the present invention, testicular self-examination is performed, one testicle at a time, by gently rolling each testicle between the thumbs, which are placed on the top of the testicle, and the index and middle fingers, which are placed behind the testicle. Palpation is often accompanied by visual inspection for any change in the size, shape, or consistency of the testes.

As used in the present application, by “muscle strain or tear” is meant damage to a muscle or its attaching tendons. Muscle strain can occur during the course of normal daily activities, with sudden heavy lifting, during sports, or while performing work tasks, and is most common, but is not limited to, the lower back, neck, shoulders, and hamstrings.

Vitamin D₃ (Cholecalciferol) has the empirical formula C₂₇H₄₄O and is available in a variety of forms from a number of suppliers raw materials used in the manufacture of personal care products, including Essential Ingredients (Lawrenceville, Ga.). Vitamin D₃ (cholecalciferol) undergoes a multi-step process, before conversion into the Calcitriol form (1,25-dihydroxyvitamin D₃), which is thought to be responsible for most of the physiological effects of vitamin D.

Precursors, derivatives and analogs of Vitamin D₃ are suitable for use in the compositions of the present invention. One non-limiting example of a Vitamin D₃ precursor suitable for use in the natural, BSE moisturizing aromatherapy creme of the present invention is 7-Dehydrocholesterol, available from MMP International (South Plainfield, N.J.). When exposed to ultraviolet radiation, 7-Dehydrocholesterol is metabolized to Vitamin D₃ in the skin, and is then further converted to its active metabolite (Calcitriol).

Vitamin E, also known as Tocopherol, has the empirical formula C₂₄H₅₀O₂. An essential fat-soluble nutrient that serves as an antioxidant and is also used in cell signaling, regulating gene expression and immune function, a wide range of derivatives of Vitamin E, including acetate, nicotinate, and other esters, are suitable for use in the natural, BSE moisturizing aromatherapy creme of the present invention. Tocopheryl acetate (empirical formula C₃₁H₅₂O₃), the ester of Tocopherol and acetic acid, is a preferred Vitamin E derivative and is available from a number of raw material suppliers, including BASF Care Chemicals (Florham Park, N.J.).

Camelia Sinensis (Green Tea) Leaf Extract, the third component of the PBH Complex, is rich in polyphenols, including catechins (which constitute up to 30% of the dry leaf weight) and their glycosides, and depsides, including chlorogenic acid and coumarylquinic acid. Four primary catechin derivatives have been isolated in Green Tea Leaf Extract—epicatechin (EC), epigallocatechin (EGC), epicatechin gallate (ECG), and epigallocatechin gallate. Green Tea Leaf Extract suitable for use in the compositions of the present invention is available from a number of specialty chemical suppliers including under the tradename Actiphyte® Japanese Green Tea Concentrate from Active Organics, A Lubrizol Company (Lewisville, Tex.).

Oenothera Biennis (Evening Primrose) Oil is an oil obtained from Oenethera biennis that is high in the omega-6 essential fatty acid, gamma-linolenic acid.

The PBH Complex consists essentially of each of four pro-breast health ingredients: (i) Vitamin E, preferably in the form of Tocopheryl Acetate; (ii) Vitamin D, preferably in the form of Vitamin D₃; (iii) Camelia Sinensis (Green Tea) Leaf Extract; and (iv) Oenothera Biennis (Evening Primrose) Oil. Preferably, each of the four pro-breast health ingredients is present at a concentration from 0.05% to 0.25% wt/wt in a creme or lotion that is formulated for topical application to the breasts. In an especially preferred embodiment, the PBH Complex consists of each of (i) Vitamin E, preferably in the form of Tocopheryl Acetate; (ii) Vitamin D, preferably in the form of Vitamin D₃; (iii) Camelia Sinensis (Green Tea) Leaf Extract; and (iv) Oenothera Biennis (Evening Primrose) Oil.

In particularly preferred embodiments, each of the four essential components of the PBH Complex is present in a creme for topical application to human female breasts at a concentration of at least 0.1% by weight, based on the total weight of the formulation.

In an even more particularly preferred embodiment, Vitamin E (or a derivative thereof), Vitamin D (or a derivative thereof), Green Tea Leaf Extract and Oenothera Biennis (Evening Primrose) Oil are each present in a creme for topical examination of the tissue underlying human skin—in particular, tissue underlying the breasts and testes, as well as muscles—in a ratio of 1:1:1:1.

As used in the present application in connection with Adjuvant Aromatherapy Blend by the term “complementary” is meant that the ingredients that comprise the Adjuvant Aromatherapy Blend produce a desired aroma, with associated sensory benefits, but do not deleteriously impact the desired activity of the four components of the PBH Complex.

In addition to the PBH Complex, natural, BSE moisturizing aromatherapy creme contains an Adjuvant Aromatherapy Blend that is complementary to the PBH Complex that contains the following three essential oils: (i) Rosa Damascena (Damask Rose) Seed Oil; (ii) Lavandula Angustifolia (Lavender) Oil; and (iii) Cymbopogon Schoenanthus (Lemongrass) Oil.

Optionally, and in a particularly preferred embodiment, the Adjuvant Aromatherapy Blend also contains a fourth essential oil, Mentha Piperita (Peppermint) Oil.

Preferably, each of the essential oils in the Adjuvant Aromatherapy Blend is present at concentration of at least 0.1% wt/wt.

A key attribute of the natural, BSE moisturizing aromatherapy creme of the present invention is a flowing, yet skin-substantive, lotion-like texture that applies, spreads easily, and does not run-off wet skin. This property is achieved in the form of an oil-in-water emulsion having a viscosity at 24 hours after formulation, as measured with a Brookfield Viscometer TE at 3 rpms, ranging from about 60,000 cps to about 100,000 cps. As will be appreciated by the person having ordinary skill in the art, the term “creme” as used in the personal care arts may also be described as a “lotion” or “cream”.

Importantly, the natural, BSE moisturizing aromatherapy creme of the present invention, is natural, by which is meant a stable emulsion is formed without the use of petroleum-based surfactants, namely ethoxylates. By stable emulsion is meant that the emulsion does not separate into its constituent oil and water phases when stored at room temperature (25° C.) for at least three months; elevated temperature (40° C.) for one month; and after three freeze-thaw cycles.

In one preferred embodiment, the natural, BSE moisturizing aromatherapy creme contains an alkyl polyglucoside, a class of non-ionic surfactants obtained from renewable, plant-derived feedstocks. Particularly preferred are APGs that are free of ethoxylates. One such especially preferred APG is PLANTAREN 2000® N UP (INCI name: Decyl Gluoside, available from BASF Care Chemicals). This fatty polyglucoside prepared by reacting cornstarch glucose with C₈-C₁₆ fatty alcohol. Preferably, PLANTAREN 2000® N UP is present in compositions of the present invention at a concentration of about 3% to about 6% wt/wt.

Other preferred alkyl polyglucosides suitable for use in the natural, BSE moisturizing aromatherapy creme of the present invention include the following, each available from BASF Care Chemicals: Caprylyl/Capryl Glucoside (tradename: PLANTACARE® 810 UP); Coco Glucoside (tradename: PLANTACARE® 818 UP); and Lauryl Glucoside (tradename: PLANTAREN® 1200 N UP).

Since visual observation is an important aspect of breast-self examination, it is important that the natural, BSE moisturizing aromatherapy creme impart a translucent, white appearance when applied to the skin. Decyl Glucoside, the preferred alkyl polyglucoside, is yellow. Accordingly, since in preferred embodiments, the present invention is natural, a stable oil-in-water emulsion is achieved by the combination of Decyl Glucoside with two organic fatty acids—myristic acid and stearic acid.

Myristic acid (empirical formula C₁₄H₂₈O₂) is preferably present in the natural, BSE moisturizing aromatherapy creme at a concentration of from about 5.5% to about 8.0% wt/wt. In a particularly preferred embodiment, myristic acid is present at a concentration of about 6.5%.

Stearic acid, CH₃(CH₂)₁₆COOH, is preferably present in the natural, BSE moisturizing aromatherapy creme at a concentration of from about 6.5% to about 9.0% wt/wt. In a particularly preferred embodiment, myristic acid is present at a concentration of about 7.5% wt/wt.

In a particularly preferred embodiment, the ratio of myristic acid to stearic acid is from about 2:3 to 3:2, and even more preferably about 5:6

In an especially preferred embodiment, the natural, BSE moisturizing aromatherapy creme also contains coconut acid having the following fatty acid distribution: caprylic acid-about 7%; capric acid-about 6%; lauric acid-about 48%; myristic acid-about 19%; palmitic acid-about 11%; stearic acid-about 3%; oleic acid-about 5%; linoleic acid-about 1%. The above coconut acid is available under the tradename Emery 622 from Emery Oleochemicals LLC (Cinncinnati, Ohio). Preferably, the coconut acid is present at a concentration of from about 0.7% to about 1.1% wt/wt, still more preferably at a concentration of about 0.9% wt/wt.

In a most preferred embodiment, in which coconut acid is present at a concentration of about 0.9% wt/wt, the natural, BSE moisturizing aromatherapy creme of the present invention contains myristic acid and stearic acid in a ratio of about 9:10.

In the natural, BSE moisturizing aromatherapy creme of the present invention, the organic fatty acids are neutralized, preferably with sodium hydroxide and/or potassium hydroxide, to produce a finished consumer product having a pH of from about 6.0 to about 7.0, one which does not cause a manifestation of a clinically-adverse side effect (e.g., significant skin irritation or sensitization). In a particularly preferred embodiment, the natural, BSE moisturizing aromatherapy creme has a pH of about 6.5 and sodium hydroxide and/or potassium hydroxide is present in a combined amount of from about 1.9 to 2.2% wt/wt.

In a preferred embodiment, the natural, BSE moisturizing aromatherapy creme is free of parabens. Preferably, the preservative is compliant with the United States National Organic Program (N.O.P.-compliant) and is classified as Generally Recognized as Safe by the United States Food and Drug Administration.

By “N.O.P.-compliant” is meant that the product complies with the requirements of Section 205.605 of Title 7 of the Code of Federal Regulations and is comprised of non-agricultural substances allowed as ingredients in processed products labeled as “organic” or “made with organic ingredients.”

A preferred N.O.P.-compliant preservative that may be used in the natural, BSE moisturizing aromatherapy creme of the present invention is Carya Ovata (Shagbark Hickory) Bark Extract. This N.O.P.-compliant preservative combination is available under the tradename ESP Safeguard WS+ from Earth Supplied Products LLC (Naples, Fla.), a supplier of raw materials for use in personal care products that is certified by the FDA as an N.O.P. Producer, Handler, and Processor. In this preferred embodiment, ESP Safeguard WS+ is present at a concentration of about 5% wt/wt.

In preferred embodiments, the natural, BSE moisturizing aromatherapy creme of the present invention contains one or more of the following Skin Health Benefit Ingredients: Sodium Hyaluronate, Hydrolyzed Wheat Protein, Chamomilla Recutita (Matricaria) Flower Extract, Agave Tequilana Stem Extract, Rosmarinus Officinalis (Rosemary) Leaf Extract, Olea Europaea (Olive) Fruit Extract, Cocos Nucifera (Coconut) Oil, Butyrospermum Parkii (Shea) Butter, Punica Granatum (Pomegranate) Extract, and Carthamus Tinctorius (Safflower) Seed Oil. Each of the above Skin Health Benefit Ingredients may, and preferably is, present at concentration of from about 0.05% to about 0.25% wt/wt, more preferably at a concentration of about 0.1% wt/wt.

In one particularly preferred embodiment, the natural, BSE moisturizing aromatherapy creme contains at least one, preferably, at least two, still more preferably at least three moisture-retaining and/or film-forming ingredients, which are not made using petroleum-based or silicone-based feedstock.

One preferred moisture-retaining ingredient, available from Infintec Activos, SL (Barcelona, Spain), is a combination of sorbitol, lecithin, xanthan gum, oleic acid, phenoxyethanol, caprylyl glycol, disodium edta, and glyceryl caprylate. This combination is sold under the tradename 3D Hydra APS, and is preferably used in the natural, BSE moisturizing aromatherapy creme at a concentration of about 1% wt/wt.

Another preferred moisture-retaining ingredient is sodium hyaluronate. In place of, or in addition to sodium hyaluronate, hyaluronic acid, or a derivative or hydrolyzate thereof, may also be used. Preferably, sodium hyaluronate is present in the natural, BSE moisturizing aromatherapy creme at a concentration of about 0.1% wt/wt.

Hydrolyzed Wheat Protein is yet another preferred film-forming/ingredient that is preferably present in the natural, BSE moisturizing aromatherapy creme at a concentration of about 0.1% wt/wt.

That testicular cancer self examination creme, which can also be used by an allied health care professional in conducting a physical examination for testicular cancer, is comprised of (i) an alkyl polyglucoside and (ii) at least one moisture-retaining and/or film-forming ingredient selected from the group consisting of (x) hyaluronic acid or a derivative thereof, (y) hydrolyzed wheat protein and (z) a mixture consisting of Sorbitol, Lecithin, Xanthan Gum, Oleic Acid, Phenoxyethanol, Caprylyl Glycol, Disodium EDTA, and Glyceryl Caprylate. This same formulation can be used by an allied health care professional in conducting a physical examination for signs of muscle strain or tear.

In one preferred aspect of these embodiments of the present invention—directed to physical examination for signs of testicular cancer or muscle tears or strain, the alkyl polyglucoside is Decyl Glucoside.

In other preferred aspect of these embodiments, the testicular cancer self-examination creme (which can also be used by an allied health professional in conducting physical examination for signs of testicular cancer or muscle strains or tears) further comprises Carya Ovata Bark Extract and/or at least two organic acids selected from the group consisting of myristic acid, stearic acid and coconut acid. In a particularly preferred aspect of these embodiments, the ratio of myristic acid to stearic acid in the moisturizing aromatherapy creme is from about 2:3 to 3:2, even more preferably about 5:6.

The following formulation example is illustrative of the natural, BSE moisturizing aromatherapy creme of the present invention. The components and specific ingredients of the example formulations are presented as being typical, and various modifications can be derived in view of the foregoing disclosure within the scope of the invention.

### Example 1

### Natural, BSE Moisturizing Aromatherapy Creme

| | | |
|---|---|---|
| Phase A | Water | 66.940 |
| | Glycerin | 5.000 |
| Phase B | Olea Europaea (Olive) Fruit Extract | 0.010 |
| | Stearic Acid | 7.500 |
| | Myristic Acid | 6.500 |
| | Coconut Acid | 0.900 |
| | Cocos Nucifera (Coconut) Oil | 0.010 |
| | Butyrospermum Parkii (Shea) Butter | 0.010 |
| Phase C | Potassium Hydroxide | 2.000 |
| | Sodium Hydroxide 10% Aq. Soln. | 0.100 |
| Phase D | Sorbitol, Lecithin, Xanthan Gum, Oleic Acid, | 1.000 |
| | Phenoxyethanol, Caprylyl Glycol, Disodium | |
| | EDTA, and Glyceryl Caprylate | |
| | Carya Ovata (Shagbark Hickory) Bark Extract | 5.000 |
| | Rosa Damascena (Damask Rose) Flower Oil | 0.010 |
| | Sodium Hyaluronate | 0.100 |
| | Lavandula Angustifolia (Lavender) Oil; | 0.100 |
| | Cymbopogon Schoenanthus (Lemongrass) Oil | |
| | Oenothera Biennis (Evening Primrose) Oil | 0.100 |
| | Mentha Piperita (Peppermint) Oil | 0.010 |
| | Hydrolyzed Wheat Protein | 0.100 |
| | Camellia Sinensis Leaf Extract | 0.100 |
| | Cholecalciferol | 0.100 |
| | Vitamin E Acetate | 0.100 |
| | Decyl Glucoside | 4.000 |
| | Punica Granatum (Pomegranate) Extract in | 0.010 |
| | Carthamus Tinctorius (Safflower) Seed Oil | |
| | Chamomilla Recutita (Matricaria) Flower Extract | 0.100 |
| | Agave Tequilana Stem Extract | 0.100 |
| | Rosmarinus Officinalis (Rosemary) Leaf Extract | 0.100 |

The formulation of Example 1 is made according to the following procedure: Into a main vessel, combine Phase A ingredients. Heat to 75-80° C.; mix until homogenous. Into a second vessel, combine Phase B (oil phase). Heat to 75-80° C.; mix until homogenous. Add Phase B mixture to Phase A mixture. Mix A/B until homogenous. Cool A/B to 40° C. Add Phase D ingredients; mix until homogenous.

### Example 2

### Breast Self-Examination

53 women, in good general health, between the ages of 20 and 75 years old, inclusive, participated in a one-day clinical study. Among the subject, all of the six Fitzpatrick Skin Types were represented. Additionally, each of the following four ethnicities were represented: at least one each of African American, Asian, Caucasian, and Hispanic. All subjects read, understood and signed an informed consent.

Subjects were provided with the BSE moisturizing aromatherapy creme of the present invention and two product performance questionnaires—the first was self-administered immediately after use; the second was administered by a researcher on the study staff after the subject completed the self-administered questionnaire. In the shower or bath, subjects were instructed to apply a liberal amount of the BSE moisturizing aromatherapy creme of the present invention to a designated damp breast, covering it completely. Subjects were cautioned not to apply the BSE moisturizing aromatherapy creme of the present invention directly under the shower spray or when immersed in bath water.

Subjects were then directed to raise one arm, dropping that arm behind their head. With the opposite hand, the subjects were instructed to hold their middle three fingers together and, using the pads of those fingers, to conduct a self-examination of the entire breast area—from the collarbone to the bottom of the rib cage, and from the armpit to the middle of the chest. Beginning at the outer breast area, subjects moved their middle three fingers around the breast in overlapping circular motions. Subjects continued making overlapping circular motions, in smaller and smaller circles, working towards the nipple, until the entire breast area had been checked. The same steps were followed for the breast, to which the BSE moisturizing aromatherapy creme of the present invention was not applied.

In the clinical trial, participants were asked to compare their ability to feel underneath the surface of the skin during breast self-examination, and to detect anything unusual, with and without using the BSE moisturizing aromatherapy creme of the present invention. Subjects were asked to rate the ability to feel underneath the surface of the skin during breast self-examination on a scale of 1-10, where 1 is not able to feel underneath the skin, and not able to detect anything unusual, and 10 is able to feel underneath the skin, and to detect anything unusual. When using the BSE moisturizing aromatherapy creme of the present invention to conduct breast self-examination, and to feel for anything unusual, 96.2% of test subjects reported an ability to feel underneath the surface of the skin during breast self-examination, with an average rating of 8.30 out of a maximum score of 10. This was statistically significant (i.e., in comparison to the same subjects reporting their ability to feel underneath the surface of the skin when conducting breast self-examination without the BSE moisturizing aromatherapy creme of the present invention).

Subjects were also asked to evaluate how freely/easily their hand moved across the surface of the skin during breast self-examination with and without using the BSE moisturizing aromatherapy creme of the present invention. Ratings were assigned on a scale of 1-10, where 1 is not easy and 10 is easiest. When using the BSE moisturizing aromatherapy creme of the present invention to conduct breast self-examination, 100% of test subjects reported that their hand moved freely/easily across the surface of the skin, with an average score of 9.36 out of 10. In marked contrast, when not using the BSE moisturizing aromatherapy creme of the present invention to conduct breast self-examination (the “untreated” group), only 36.5% of study participants reported that their hand moved freely/easily across the surface of the skin. Participants in the untreated group rated their ability to move their hand freely/easily across the surface of the skin on average as 5.96 on the same 1-10 scale. This difference was statistically significant.

Additionally, based on a dansyl chloride absorption test, the BSE moisturizing aromatherapy creme of the present invention was determined to be immediately absorbed into the skin.

Use of dansyl chloride accompanied by tape stripping is a test method well known to the person of ordinary skill in the arts of dermatology and topical formulation. Typically, dansyl chloride is mixed with a test product, which is applied and rubbed into the skin. The test product is then removed, the site of application dried, and layers of skin from the test site are removed with adhesive tape. When placed under a Wood's lamp, a strip containing dansyl chloride fluoresces. See, e.g., M. Takahashi, “Measurement of Turnover Time of Stratum Corneum Using Dansyl Chloride Fluorescence,”J. Soc. Cosmet. Chem. Vol. 38, pp. 321-331 (September/October 1987).

In the study of Example 2, dansyl chloride was mixed with the BSE moisturizing aromatherapy creme of the present invention. More particularly, using a metered pipette, a technician applied approximately 100 μL of the test product—the BSE moisturizing aromatherapy creme into which dansyl chloride had been added—to a test site (2×2 cm²) on the volar forearm of each subject. The product was dotted evenly throughout the test site, and was then gently rubbed into the test site using a gloved finger. Subjects then rinsed their forearm under tepid running water for fifteen seconds and patted the forearm dry. Up to ten tape strips were applied and successively removed from the site of application. Each strip was placed under a Wood's light and examined for fluorescence—indicating the presence of dansyl chloride. Dansyl chloride fluorescence was observed in 100% of strips 1-7, 98.1% of strip 8, 94.3% of strip 9, and 90.5% of strip 10.

As used in the describing the compositions and methods of the present invention, by “immediately absorbed into the skin” is meant that after application to the skin—namely, a 2×2 cm²) on the volar forearm—fluorescence from dansyl chloride is observed under a Wood's light after at least five tape strippings.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove, but rather that the claims be construed as encompassing all the features of patentable novelty which reside in the present invention, including all features which would be treated as equivalents thereof by those skilled in the art to which the invention pertains.

1. A natural, breast examination moisturizing aromatherapy creme comprising
(a) a “Pro Breast Health Complex” consisting essentially of
(i) Green Tea Extract,
(ii) Vitamin D, or a derivative thereof,
(iii) Vitamin E, or a derivative thereof, and
(iv) Oenothera Biennis (Evening Primrose) Oil; and
(b) an Adjuvant Aromatherapy Blend that is complementary to the PBH Complex that comprises
(i) Rosa Damascena (Damask Rose) Seed Oil,
(ii) Lavandula Angustifolia (Lavender) Oil, and
(iii) Cymbopogon Schoenanthus (Lemongrass) Oil;
wherein the natural, breast examination moisturizing aromatherapy creme does not contain any parabens and does not contain petroleum-based surfactants.

2. The natural, breast examination moisturizing aromatherapy creme of claim 1, further comprising an alkyl polyglucoside.

3. The natural breast examination moisturizing aromatherapy creme of claim 2 wherein the alkyl polyglucoside is Decyl Glucoside.

4. The natural, breast examination moisturizing aromatherapy creme of claim 2, further comprising Carya Ovata Bark Extract.

5. The natural, breast examination moisturizing aromatherapy creme of claim 4, further comprising at least one moisture-retaining and/or film-forming ingredient selected from the group consisting of (a) hyaluronic acid or a derivative thereof, (b) hydrolyzed wheat protein and (c) a mixture consisting of Sorbitol, Lecithin, Xanthan Gum, Oleic Acid, Phenoxyethanol, Caprylyl Glycol, Disodium EDTA, and Glyceryl Caprylate.

6. The natural, breast examination moisturizing aromatherapy creme of claim 2, further comprising at least two organic acids selected from the group consisting of myristic acid, stearic acid and coconut acid.

7. The natural, breast examination moisturizing aromatherapy creme of claim 6, wherein the ratio of myristic acid to stearic acid is from about 2:3 to about 3:2.

8. The natural, breast examination moisturizing aromatherapy creme of claim 1, wherein the creme is applied by a woman to her breasts and underarm areas for the propose of conducting a breast self-examination.

9. A method of conducting breast self-examination comprising the steps of (a) applying a natural, breast examination moisturizing aromatherapy creme according to claim 1 and (b) palpating the breasts and underarm areas to detect a lump, nodule, knot or thickening of tissue.

10. The method of claim 9 wherein the woman applies the natural, breast examination moisturizing aromatherapy creme to wet skin, in or immediately after a shower or bath.

11. The method of claim 10, wherein the natural breast examination moisturizing aromatherapy is immediately absorbed into the skin.

12. The natural breast examination moisturizing aromatherapy creme of claim 1, wherein the Pro Breast Health Complex consists of four components:
(i) Green Tea Extract;
(ii) Vitamin D, or a derivative thereof;
(iii) Vitamin E, or a derivative thereof; and
(iv) Oenothera Biennis (Evening Primrose) Oil.

13. A method of conducting physical examination of the testes for signs of testicular cancer comprising the steps of
(a) applying to the scrotum a physical examination creme that does not contain any parabens and does not contain petroleum-based surfactant, wherein the creme comprises
(i) an alkyl polyglucoside and
(ii) at least one moisture-retaining and/or film-forming ingredient selected from the group consisting of (x) hyaluronic acid or a derivative thereof, (y) hydrolyzed wheat protein and (z) a mixture consisting of Sorbitol, Lecithin, Xanthan Gum, Oleic Acid, Phenoxyethanol, Caprylyl Glycol, Disodium EDTA, and Glyceryl Caprylate and
(b) palpating the testicles to detect a lump, nodule, knot or thickening of tissue.

14. The method of claim 13 wherein the alkyl polyglucoside is Decyl Glucoside.

15. The method of claim 13 wherein the physical examination creme further comprises Carya Ovata Bark Extract.

16. The method of claim 13 wherein the physical examination creme further comprises at least two organic acids selected from the group consisting of myristic acid, stearic acid and coconut acid.

17. The method of claim 16 wherein the ratio of myristic acid to stearic acid in the physical examination creme is from about 2:3 to about 3:2.

18. A method of conducting physical examination for signs of muscle tears or strains comprising the steps of
(a) applying to the skin above the muscle that may be torn or strained a physical examination creme that does not contain any parabens and does not contain petroleum-based surfactant, wherein the creme comprises
(i) an alkyl polyglucoside and
(ii) at least one moisture-retaining and/or film-forming ingredient selected from the group consisting of (x) hyaluronic acid or a derivative thereof, (y) hydrolyzed wheat protein and (z) a mixture consisting of Sorbitol, Lecithin, Xanthan Gum, Oleic Acid, Phenoxyethanol, Caprylyl Glycol, Disodium EDTA, and Glyceryl Caprylate and
(b) palpating the muscle tissue underneath the skin to detect a strain or tear.

19. A topically-applied, pro-breast health complex consisting of (i) Green Tea Extract, (ii) Vitamin D, or a derivative thereof, (iii) Vitamin E, or a derivative thereof, and (iv) Oenothera Biennis (Evening Primrose) Oil.
